(19) United States
(12) Patent Application Publication
(10) Pub. No.: US 2015/0147309 A1
(43) Pub. Date: May 28, 2015

(54) ALLOSTERIC CHAPERONES AND USES THEREOF

(71) Applicants: Fondazione Telethon, Roma (RM) (IT); Consiglio Nazionale delle Ricerche, Roma (RM) (IT)

(72) Inventors: Giancarlo Parenti, Napoli (NA) (IT); Caterina Porto, Napoli (NA) (IT); Marco Moracci, Napoli (NA) (IT); Maria Carmina Ferrara, Napoli (NA) (IT); Beatrice Cobucci-Ponzano, Napoli (NA) (IT); Generoso Andria, Napoli (NA) (IT)

(21) Appl. No.: 14/405,575

(22) PCT Filed: Jun. 6, 2013
(86) PCT No.: PCT/EP2013/061730

§ 371 Date: Dec. 4, 2014

Related U.S. Application Data
(60) Provisional application No. 61/656,246, filed on Jun. 6, 2012.

(51) Int. Cl. A61K 31/4172; A61K 38/47; G01N 33/573; A61K 31/40; A61K 31/405; A61K 31/195; A61K 31/401

(52) U.S. Cl. CPC A61K 31/4172; A61K 31/195; A61K 38/47; A61K 31/401; A61K 31/40; A61K 31/405; G01N 33/573; G01N 2500/04; G01N 2333/928
USPC 424/94.61; 514/563; 514/562; 514/400; 514/423; 514/419; 435/7.4; 435/18

(57) ABSTRACT

The present invention relates to an allosteric non-inhibitory chaperone of the lysosomal acid alpha-glucosidase (GAA) for use in the treatment of a pathological condition characterized by a deficiency of the lysosomal acid alpha-glucosidase (GAA), to pharmaceutical composition thereof, to a method for increasing the activity of GAA in a subject and to a method for identifying an allosteric non-inhibitory chaperone for GAA.

### FIELD OF THE INVENTION

The present invention relates to an allosteric non-inhibitory chaperone of the lysosomal acid alpha-glucosidase (GAA) for use in the treatment of a pathological condition characterized by a deficiency of the lysosomal acid alpha-glucosidase (GAA), to pharmaceutical composition thereof, to a method for increasing the activity of GAA in a subject and to a method for identifying an allosteric non-inhibitory chaperone for GAA.

### BACKGROUND OF THE INVENTION

Pompe disease (PD) is an inherited metabolic disorder due to the deficiency of the lysosomal acid alpha-glucosidase (GAA). The disease manifestations, due to glycogen accumulation, are highly debilitating and are predominantly related to the involvement of heart, with a severe hypertrophic cardiomyopathy, and of skeletal muscles, with progressive motor impairment.

Pompe disease (PD, OMIM 232300) is an inborn metabolic disorder caused by the functional deficiency of alpha-glucosidase (GAA, acid maltase, E.C.3.2.1.20), an acid glycoside hydrolase involved in the lysosomal breakdown of glycogen. GAA deficiency results in glycogen accumulation in lysosomes and in secondary cellular damage, with mechanisms not fully understood [van der Ploeg and Reuser 2008; Shea and Raben, 2009; Parenti and Andria, 2011]. Although GAA deficiency in PD is generalized, muscles are particularly vulnerable to glycogen storage. The disease manifestations are thus predominantly related to the involvement of cardiac and skeletal muscles. The phenotypic spectrum is wide and varies from the devastating classical infantile-onset form of the disease, characterized by severe cardiomyopathy, feeding difficulties, respiratory infections and early lethality, to attenuated phenotypes characterized by later onset (childhood, juvenile or adult) and absent or mild cardiac involvement. The manifestations related to progressive muscle hypotonia, causing severe motor impairment and eventually respiratory failure, impact severely on the health of PD patients [van der Ploeg and Reuser, 2008].

The only approved treatment for PD, enzyme replacement therapy (ERT) with recombinant human GAA (rhGAA), has shown limited therapeutic efficacy in some patients, suggesting that further innovative approaches for the treatment of PD patients would be desirable. Among these, pharmacological chaperone therapy (PCT) represents a promising strategy. However, the chaperones so far identified also have limitations, particularly because of their potential inhibitory effects on target enzyme, GAA.

Enzyme replacement therapy (ERT) with recombinant human glucosidase alpha (rhGAA) is currently considered the standard of care for the treatment of this disorder. Several studies support the efficacy of this approach in improving survival and function or in stabilizing the disease course [van den Hout et al, 2000; van den Hout et al, 2004; Kishnani et al, 2007; Strothotte et al, 2010; van der Ploeg et al, 2010]. However, other reports suggest that ERT in PD suffers from limitations [Schoser et al, 2008]. Despite treatment, some patients experience little clinical benefit or show signs of disease progression. It is also clear that reaching therapeutic concentrations of the recombinant enzyme in skeletal muscle is particularly challenging.

Several factors concur in limiting therapeutic success of ERT. Some of them are related to clinical aspects of the disease, such as the age at start of treatment [Chien et al, 2009; Kishnani et al, 2009], the presence of irreversible cellular and tissue damage, the immunological and cross-reactive material (CRIM) status of patients [Kishnani et al, 2010], the need of invasive procedures for enzyme administration (frequent intravenous infusions or permanent intravenous devices), and the high costs of therapy [Beutler, 2006]. Other factors are related to the cellular biology of the disease and to the targeting and trafficking of the recombinant enzyme. These include the preferential uptake of rhGAA by liver and the insufficient targeting of the enzyme to skeletal muscle [Raben et al, 2003], the relative deficiency of the cation-independent mannose-6-phosphate receptor (CI-MPR) in muscle cells [Wenk et al, 1991; Koeberl et al, 2011] and its abnormal cellular distribution [Cardone et al, 2008], the “build up” of the autophagic compartment observed in myocytes [Fukuda et al, 2006; Fukuda et al, 2006; Raben et al, 2009].

In addition, studies in other LSDs treatable by ERT, such as Gaucher disease (due to the deficiency of beta-glucocerebrosidase, GBA), point to the role of factors intrinsically related to the recombinant enzymes used for ERT, and suggest that these enzymes may be relatively unstable when exposed to stresses, like non-acidic pH, during their transit to lysosomes [Xu et al, 1996; Shen et al, 2008; Benjamin et al, 2012].

For all these reasons strategies directed towards the improvement of the therapeutic action of ERT in PD, or to the identification of alternative approaches for the treatment of this disorder would be highly desirable.

An approach that has been proposed in the recent years is pharmacological chaperone therapy (PCT). This approach has been designed for the treatment of diseases due to protein misfolding, by using small-molecule ligands that increase stability of mutated proteins and prevent their degradation [Fan, 2008; Parenti, 2009; Valenzano et al, 2011]. Recent studies, however, have shown that chaperones are not only able to rescue misfolded defective proteins, but may also potentiate the effects of the wild-type recombinant enzymes used for ERT. Authors and others provided pre-clinical evidence supporting this concept in studies showing synergy between chaperones and ERT in two among the most prevalent lysosomal disorders, i.e. PD [Porto et al, 2009] and Fabry disease [Porto et al, 2011; Benjamin et al, 2012]. In both disorders, when recombinant enzymes were administered to mutant fibroblasts in combination with the chaperone molecules N-butyl-deoxynojirimycin (NB-DNJ) and 1-deoxy-galactonojiirimycin (DGJ), respectively, the lysosomal trafficking, the maturation and the intracellular activity of the enzymes increased. Improved stability of rhGAA was also observed in PD fibroblasts [Porto et al, 2009]. A similar effect was obtained also in cultured macrophages treated with the recombinant GBA (the enzyme used for ERT in Gaucher disease), in the presence of the chaperone isofagomine (IFG) [Shen et al, 2008].

Compared to ERT, small-molecule chaperones have important advantages in terms of biodistribution, oral availability, reduced impact on patients' quality of life. However, some aspects of their action remain poorly characterized. Specifically, information is still lacking on their intracellular distribution, inhibition of resident enzymes in specific cellular compartments, and specificity. A reason of major concern on the clinical use of these drugs is that all the chaperones so far identified for the treatment of LSDs are reversible competitive inhibitors of the target enzymes, named also as “active site-directed” chaperone, as they bind the active site of the target enzyme [Valenzano et al, 2011].

In this respect, the identification of second-generation chaperones may be advantageous. An ideal chaperone should be able to protect the enzymes from degradation without interfering with its activity, be largely bioavailable in tissues and organs, reach therapeutic levels in cellular compartments where its therapeutic action is required, show high specificity for the target enzyme with negligible effects on other enzymes, and have a good safety profile. Drugs already approved for human therapy would be most advantageous for rapid clinical translation (without the need for phase I clinical trials). Extensive search for new chaperones is currently being done by high-throughput screenings with chemical libraries [Tropak et al, 2007; Zheng et al, 2007; Urban et al, 2008].

US2006115376 discloses the use of NAC as a stabilizer in a method for sterilizing a preparation of one or more digestive enzymes that is sensitive to radiation.

WO97/16170 discloses a method of treatment or prevention of a coronary condition comprising:

a) providing a first pharmaceutical composition comprising a therapeutically effective amount of a first therapeutic agent, and
b) administering the first pharmaceutical composition to the pericardial space of a patient. The first therapeutic agent can be NAC metal chelator and anti-oxidant, because of its activity as inhibitor of NFkB.

WO2004/093995 describes the use of NAC as an agent which increases the levels of oxidant defenses and/or at least one antioxidant in a human or non-human animal, in the manufacture of a medicament for treating or preventing a bone loss disorder in the human or non-human animal.

It was not known from the prior art that NAC could act as allosteric non-inhibitory chaperone for lysosomal acid alpha-glucosidase (GAA), thus being useful in pharmacological chaperone therapy (PCT).

### SUMMARY OF THE INVENTION

In the present invention the authors characterized the effects of novel allosteric non-inhibitory chaperone of the lysosomal acid alpha-glucosidase (GAA) on GAA. In particular, N-acetyl cysteine (NAC) and two related compounds (N-acetyl serine, NAS; N-acetyl glycine, NAG) were studied. Authors found that these drugs were able to stabilize wild type GAA at non-lysosomal pH, to enhance the residual activity of mutated GAA and to improve the efficacy of recombinant GAA, in particular rhGAA, used for ERT in pathological conditions characterized by a deficiency of the lysosomal acid alpha-glucosidase (GAA), such as Pompe disease. These novel chaperones did not interact with the GAA catalytic domain, and consequently were not competitive inhibitors of the enzyme. In this respect, and NAC being a molecule already approved for clinical use, these drugs may represent a significant advancement and provide a new tool for the treatment of PD. The molecules were also able to improve thermal stability of the enzyme without disrupting its catalytic activity thereby not interacting with the GAA catalytic domain. Thus, unlike the known chaperones for GAA N-butyl-deoxynojirimycin (NB-DNJ) and 1-deoxy-nojiirimycin (DNJ), NAC is not a competitive inhibitor of the enzyme. NAC also enhanced the residual activity of mutated GAA, both in cultured fibroblasts from five PD patients and in COST cells over-expressing mutated GAA gene constructs. Remarkably, NAC greatly improved the efficacy of recombinant GAA, in particular rhGAA, in PD fibroblasts incubated with the chaperone and with the recombinant enzyme, with 3.7 to 13.0-fold increases of the activity obtained with rhGAA alone. This synergistic effect of NAC and rhGAA effect has the potential to translate into improved therapeutic efficacy of ERT in PD.

It is therefore an object of the invention an allosteric non-inhibitory chaperone of the lysosomal acid alpha-glucosidase (GAA) for use in the treatment of a pathological condition characterized by a deficiency of the lysosomal acid alpha-glucosidase (GAA).

Preferably, the allosteric non-inhibitory chaperone is a N-acetylated amino acid. Still preferably the allosteric non-inhibitory chaperone is selected from the group consisting of: N-acetyl cysteine (NAC), N-acetyl serine (NAS) or N-acetyl glycine (NAG).

In a preferred embodiment the pathological condition is a lysosomal storage disease. Still preferably the lysosomal storage disease is Pompe disease (PD).

It is a further object of the invention a pharmaceutical composition comprising at least one allosteric non-inhibitory chaperone of the lysosomal acid alpha-glucosidase (GAA) and pharmaceutically acceptable excipients.

It is a further object of the invention a pharmaceutical composition comprising at least one allosteric non-inhibitory chaperone of the lysosomal acid alpha-glucosidase (GAA), a recombinant GAA and pharmaceutically acceptable excipients.

Preferably, the pharmaceutical compositions of the invention further comprise an “active site-directed” chaperone.

It is a further object of the invention a pharmaceutical composition comprising at least one allosteric non-inhibitory chaperone of the lysosomal acid alpha-glucosidase (GAA), an “active site-directed” chaperone and pharmaceutically acceptable excipients.

Preferably, the allosteric non-inhibitory chaperone is a N-acetylated amino acid.

Still preferably the allosteric non-inhibitory chaperone is selected from the group consisting of: N-acetyl cysteine (NAC), N-acetyl serine (NAS) or N-acetyl glycine (NAG).

In a preferred embodiment the “active site-directed” chaperone is selected from the group consisting of: N-butyl-deoxynojirimycin (NB-DNJ) or 1-deoxy-nojiirimycin (DNJ).

In a preferred embodiment the pharmaceutical compositions of the invention are for oral or intravenous administration.

It is a further object of the invention a method of treatment of a pathological condition characterized by a deficiency of the lysosomal acid alpha-glucosidase (GAA) comprising the administration of an effective dose of an allosteric non-inhibitory chaperone of the lysosomal acid alpha-glucosidase (GAA) to a patient in need thereof.

Preferably the pathological condition characterized by a deficiency of the lysosomal acid alpha-glucosidase (GAA) is a lysosomal storage disease.

More preferably the lysosomal storage disease is Pompe disease (PD).

In the above method, preferably the allosteric non-inhibitory chaperone is a N-acetylated amino acid. More preferably the allosteric non-inhibitory chaperone is selected from the group consisting of: N-acetyl cysteine (NAC), N-acetyl serine (NAS) or N-acetyl glycine (NAG).

The above method preferably further comprises the administration of an effective amount of exogenous GAA and/or the administration of an effective amount of an “active site-directed” chaperone.

Preferably the “active site-directed” chaperone is selected from the group consisting of: N-butyl-deoxynojirimycin (NB-DNJ) or 1-deoxy-nojiirimycin (DNJ).

It is a further object of the invention a method for increasing the activity of an endogenous and/or exogenous GAA in an individual suspected of suffering or suffering from a pathological condition characterized by a deficiency of the lysosomal acid alpha-glucosidase (GAA), which comprises administering to the individual an allosteric non-inhibitory chaperone of the lysosomal acid alpha-glucosidase (GAA) in an amount effective to increase activity of the endogenous and/or exogenous GAA in the individual.

Preferably the endogenous GAA is in a wild type or mutant form and the exogenous GAA is a recombinant GAA. The endogenous GAA is the enzyme present in the body of the subject while exogenous GAA is prepared outside of the subject and is administered to the subject.

In the above method preferably the pathological condition characterized by a deficiency of the lysosomal acid alpha-glucosidase (GAA) is a lysosomal storage disease, still preferably Pompe disease.

In the above method preferably the allosteric non-inhibitory chaperone is a N-acetylated amino acid.

Still preferably the allosteric non-inhibitory chaperone is selected from the group consisting of: N-acetyl cysteine (NAC), N-acetyl serine (NAS) or N-acetyl glycine (NAG).

In a preferred embodiment the allosteric non-inhibitory chaperone stabilizes wild type lysosomal acid alpha-glucosidase (GAA) at non-lysosomal pH, preferably at pH 7.0 and/or enhances the residual activity of mutated GAA and/or stabilizes exogenous GAA at non-lysosomal pH, preferably at pH 7.0 and/or improves the efficacy of exogenous GAA.

It is a further object of the invention the use of NAC and/or NAS and/or NAG chaperone labeled with a marker to identify an allosteric non-inhibitory chaperone for GAA.

The marker maybe a fluorescent or luminescent marker.

It is a further object of the invention a method for identifying an allosteric non-inhibitory chaperone for GAA comprising the steps of:

a) labelling NAC and/or NAS and/or NAG chaperone with a fluorophore;
b) adding to said labeled NAC and/or NAS and/or NAG an amount of rhGAA to obtain a basal rhGAA fluorescence;
c) measuring the basal rhGAA fluorescence;
d) adding a test agent;
e) measuring the fluorescence of rhGAA;
f) comparing the fluorescence of rhGAA measured in c) and e);
wherein if a variation of intensity of fluorescence or if a variation of wavelength of fluorescence is observed then the test agent is an allosteric non-inhibitory chaperone for GAA.

In the present invention an allosteric non-inhibitory chaperone of the lysosomal acid alpha-glucosidase (GAA) is a molecule that stabilizes wild type GAA at non-lysosomal pH (lysosomal pH is about 5.2, then non-lysosomal pH is a pH different from pH 5.0, for example, pH 7.0) and/or enhances the residual activity of mutated GAA and/or stabilizes recombinant GAA at non-lysosomal pH (i.e. at pH different from pH 5.0, for example pH 7.0) and/or improves the efficacy of recombinant GAA. Further the molecule does not interact with the GAA catalytic domain, and consequently is not a competitive inhibitor of the enzyme. The molecules are also able to improve thermal stability of the enzyme without disrupting its catalytic activity thereby not interacting with the GAA catalytic domain.

In the present invention a N-acetylated amino acid is any D or L N-acetylated amino acid. The N-acetylated amino acid may be any proteinogenic (natural) D/L N-acetylated amino acid or any non-natural D/L N-acetylated amino acid. Examples of natural amino acids are shown in Table I.

TABLE I
N-acetylated proteinogenic D/L-amino acids
N-acetylated proteinogenic D/L-amino acids

| Name | Structure |
|---|---|
| N-acetyl- Alanine | 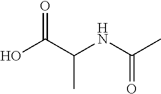 |
| N-acetyl- Arginine | 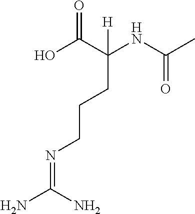 |
| N-acetyl- Asparagine | 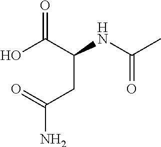 |
| N-acetyl- Aspartic Acid | 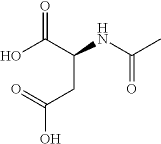 |
| N-acetyl- Cysteine | 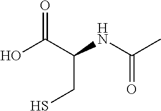 |
| N-acetyl- Glutamic acid | 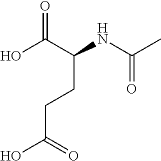 |
| N-acetyl- Glutamine | 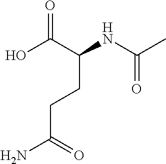 |
| N-acetyl- Glycine | 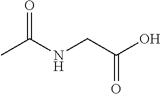 |
| N-acetyl- istidine | 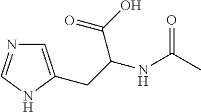 |
| N-acetyl- Isoleucine | 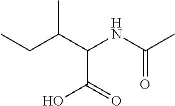 |
| N-acetyl- Leucine | 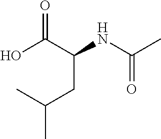 |
| N-acetyl- Lysine | 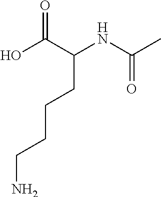 |
| N-acetyl- Methionine | 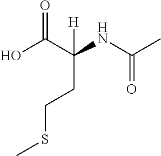 |
| N-acetyl- Phenylalanine | 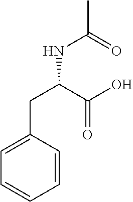 |
| N-acetyl- Proline | 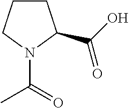 |
| N-acetyl- Pyrrolysine | 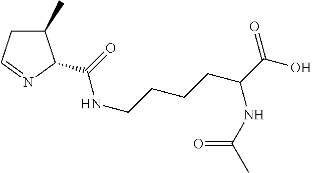 [SMILES: CC(=O)NC(CCCCNC(=O)[C@@H]1N=CC[C@H]1C)C(=O)O] |
| N-acetyl- Selenocysteine | 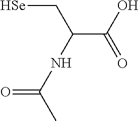 |
| N-acetyl- Serine | 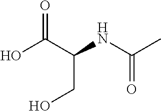 |
| N-acetyl- Threonine | 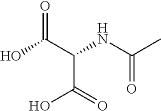 |
| N-acetyl- Tryptophan | 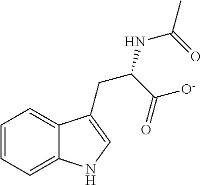 [SMILES: CC(=O)N[C@@H](Cc1c[nH]c2ccccc12)C(=O)[O-]] |
| N-acetyl- Tyrosine | 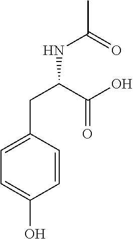 [SMILES: CC(=O)N[C@@H](Cc1ccc(O)cc1)C(=O)O] |
| N-acetyl- Valine | 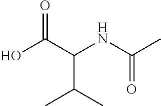 |

Non proteinogenic D/L amino acids are for instance 2-aminoisobutyric acid, ornithine, citrulline, lanthionine, djenkolic acid, diaminopimelic acid, norvaline, norleucine, homonorleucine, hydroxyproline. Examples of N-acetylated non proteinogenic amino acids are shown in Table II.

TABLE II
Examples of N-acetylated non proteinogenic D/L-amino acids.
N-acetylated non proteinogenic D/L-amino acids

| Name | Structure |
|---|---|
| N-acetyl-2- Aminoisobutyric acid | 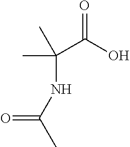 |
| N-acetyl- Ornithine | 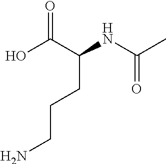 |
| N-acetyl- Citrulline | 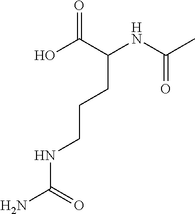 |
| N-acetyl- Lanthionine | 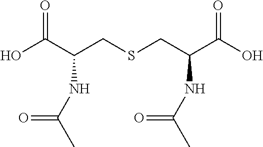 [SMILES: CC(=O)N[C@@H](CSC[C@H](NC(C)=O)C(=O)O)C(=O)O] |
| N-acetyl- Djenkolic acid | 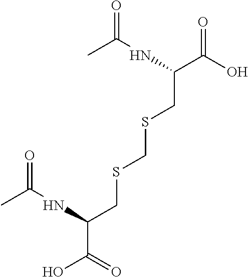 [SMILES: CC(=O)N[C@@H](CSCSC[C@H](NC(C)=O)C(=O)O)C(=O)O] |
| N-acetyl- Diaminopimelic acid | 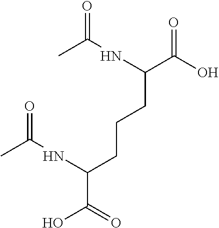 [SMILES: CC(=O)NC(CCCC(NC(C)=O)C(=O)O)C(=O)O] |
| N-acetyl- norvaline | 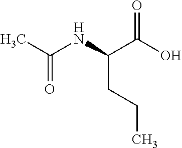 |
| N-acetyl- Norleucine | 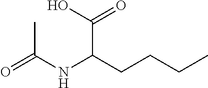 |
| N-acetyl- Homonorleucine | 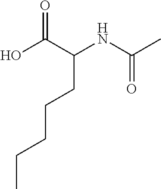 |
| N-acetyl- Hydroxyproline | 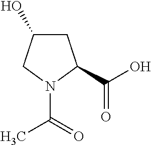 |

Such non natural amino acids are also disclosed in Unnatural Amino Acids, Methods and Protocols Series: Methods in Molecular Biology, Vol. 794, Pollegioni, Loredano; Servi, Stefano (Eds. 2012, XIV, 409p. 123 illus., Humana Press) and are part of the present invention.

Further, the skilled person in the art may identify other non natural amino acids based on their biological activity and they are also part of the present invention.

It is an object of the present invention a pharmaceutical composition comprising a therapeutically effective amount of at least one allosteric non-inhibitory chaperone as defined above and suitable diluents and/or excipients and/or adjuvants and/or emollients. The pharmaceutical composition is used for the prophylaxis and/or treatment of a pathological condition characterized by a deficiency of the lysosomal acid alpha glucosidase (GAA) as defined above. These pharmaceutical compositions can be formulated in combination with pharmaceutically acceptable carriers, excipients, stabilizers, diluents or biologically compatible vehicles suitable for administration to a subject (for example, physiological saline). Pharmaceutical composition of the invention include all compositions wherein said compounds are contained in therapeutically effective amount, that is, an amount effective to achieve the medically desirable result in the treated subject. The pharmaceutical compositions may be formulated in any acceptable way to meet the needs of the mode of administration. The use of biomaterials and other polymers for drug delivery, as well the different techniques and models to validate a specific mode of administration, are disclosed in literature. Any accepted mode of administration can be used and determined by those skilled in the art. For example, administration may be by various parenteral routes such as subcutaneous, intravenous, intradermal, intramuscular, intraperitoneal, intranasal, transdermal, oral, or buccal routes. Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyloleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99 percent, preferably from about 20 to 75 percent of active compound together with the excipient. Compositions which can be administered rectally include suppositories. It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. The compounds of the present invention may be administered to the patient intravenously in a pharmaceutical acceptable carrier such as physiological saline. Standard methods for intracellular delivery of peptides can be used, e.g. delivery via liposomes. Such methods are well known to those of ordinary skill in the art. The formulations of this invention are useful for parenteral administration, such as intravenous, subcutaneous, intramuscular, and intraperitoneal. As well known in the medical arts, dosages for any one patient depends upon many factors, including the patient's size, body surface area, age, the particular compound to be administered, sex, time and route of administration, general health, and other drugs being administered concurrently.

For a therapy comprising the administration of a allosteric non-inhibitory chaperone of the lysosomal acid alpha-glucosidase (GAA) as defined above, the persons of skill in the art will understand that an effective amount of the compounds used in the methods of the invention can be determined by routine experimentation, but is expected to be an amount resulting in serum levels between 5 and 10 mM. The effective dose of the compounds is expected to be between 100 and 1000 mg/kg body weight/day. The compounds can be administered alone or optionally along with pharmaceutically acceptable carriers and excipients, in preformulated dosages. The administration of an effective amount of the compound will result in an increase in the lysosomal enzymatic activity in the cells and tissues of a patient sufficient to improve the symptoms of the disease. For a combined therapy comprising the administration of a allosteric non-inhibitory chaperone of the lysosomal acid alpha-glucosidase (GAA) as defined above and a GAA, preferably recombinant, preferred dosages of the compounds in a combination therapy of the invention are also readily determined by the skilled artisan. Such dosages may range from 100 to 1000 mg/kg body weight/day. The administration of an effective amount of the compound will result in an improved correction of alfa-glucosidase activity by enzyme replacement therapy with recombinant human alfa-glucosidase in the cells and tissues of a patient sufficient to improve the symptoms of the disease.

In a preferred embodiment, the combination therapy comprises administration once every week or once every two weeks.

For the combination of allosteric chaperones and an active site-directed chaperone preferred dosages of the compounds in a combination therapy of the invention are also readily determined by the skilled artisan. Such dosages may range from 100 to 1000 mg/kg body weight/day for each compound in a combination therapy. In the case of N-butyl deoxynojirimycin the doses already approved for human therapy correspond to 250 mg/m2 body surface.

In the present invention molecule labelling can be made with methods know to the skilled in the art, e.g. chemical methods or methods commercially available including thiol-, amine-, N-terminal-, and C-terminal labelling, and by using the different fluorophores commercially available.

The protective effect of ligands putatively binding to allosteric site(s) are analyzed by comparing the fluorescence of the labelled enzyme.

The binding of ligands competing with allosteric non-inhibitory chaperone of the lysosomal acid alpha-glucosidase (GAA) for the allosteric site are followed by comparing the fluorescence GAA bound to allosteric non-inhibitory chaperone of the lysosomal acid alpha-glucosidase (GAA) labelled with specific fluorophores in the presence and absence of the ligand.

The allosteric non-inhibitory chaperone of the lysosomal acid alpha-glucosidase (GAA) is preferably N-acetyl cysteine (NAC), N-acetyl serine (NAS) or N-acetyl glycine (NAG). Illustrative examples of the above method include: fluorescence assays exploiting NAC/NAS/NAG labelled with specific fluorophores and/or rhGAA thiol-, amine-, N-terminal-, and C-terminal labelled with different fluorophores. In these assays, the thermal/pH stability of rhGAA are analysed by kinetics and equilibria of denaturation by following the fluorescence of the enzyme labelled with different probes. The protective effect at these conditions of ligands putatively binding to allosteric site(s) are analysed by comparing the fluorescence of the labelled enzyme. Moreover, the binding of ligands putatively binding to NAC/NAS/NAG allosteric site are followed by comparing the fluorescence of NAC/NAS/NAG labelled with specific fluorophores in the presence and absence of the ligand.

These drugs were able to stabilize wild type GAA at neutral pH (7.0), and to improve thermal stability of the enzyme without disrupting its catalytic activity thereby not interacting with the GAA catalytic domain. Thus, unlike the known chaperones for GAA N-butyl-deoxynojirimycin (NB-DNJ) and 1-deoxy-nojiirimycin (DNJ), NAC is not a competitive inhibitor of the enzyme.

In the present invention a lysosomal storage disease may be: activator deficiency/GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, Gaucher disease (including Type I, Type II, and Type III), GM1 gangliosidosis (including infantile, late infantile/juvenile, adult/chronic), I-cell disease/mucolipidosis II, infantile free sialic acid storage disease/ISSD, juvenile hexosaminidase A deficiency, Krabbe disease (including infantile onset, late onset), metachromatic leukodystrophy, pseudo-Hurler polydystrohpy/mucolipidosis IIIA, MPS I Hurler syndrome, MPS I Scheie syndrome, MPS I Hurler-Scheie syndrome, MPS II Hunter syndrome, Sanfilippo syndrome type A/MPS IIIA, Sanfilippo syndrome type B/MPS IIIB, Morquio type A/MPS WA, Morquio Type B/MPS IVB, MPS IX hyaluronidase deficiency, Niemann-Pick disease (including Type A, Type B, and Type C), neuronal ceroidlipofuscinoses (including CLN6 disease, atypical late infantile, late onset variant, early juvenile Baten-Spielmeyer-Vogt/juvenile NCL/CLN3 disease, Finnish variant late infantile CLN5, Jansky-Bielschowsky disease/late infantile CLN2/TPP1 disease, Kufs/adult-onset NCL/CLN4 disease, northern epilepsy/variant late infantile CLN8, and Santavuori-Haltia/infantile CLN1/PPT disease), beta-mannosidosis, Pompe disease/glycogen storage disease type II, pycnodysostosis, Sandhoff disease/adult onset/GM2 gangliosidosis, Sandhoff disease/GM2 gangliosidosis infantile, Sandhoff disease/GM2 gangliosidosis juvenile, Schindler disease, Salla disease/sialic acid storage disease, Tay-Sachs/GM2 gangliosidosis, Wolman disease, Multiple Sulfatase Deficiency.

The invention will be described now by non-limiting examples referring to the following figures.

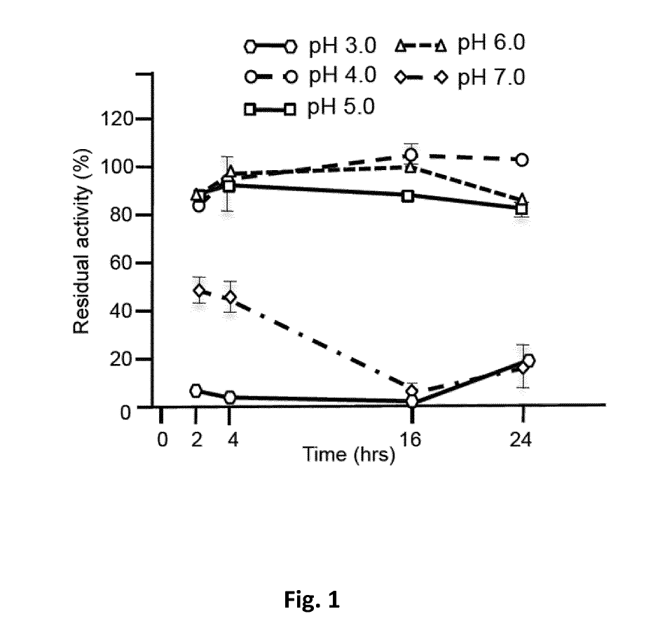

FIG. 1. Effect of pH on rhGAA. rhGAA was kept at 37° C. for variable times (0 to 24 hrs) at both acidic (3.0, 4.0, 5.0, 6.0) and neutral (7.0) pH. The residual activity refers to rhGAA incubated in its storage buffer for the same time and assayed in standard conditions.

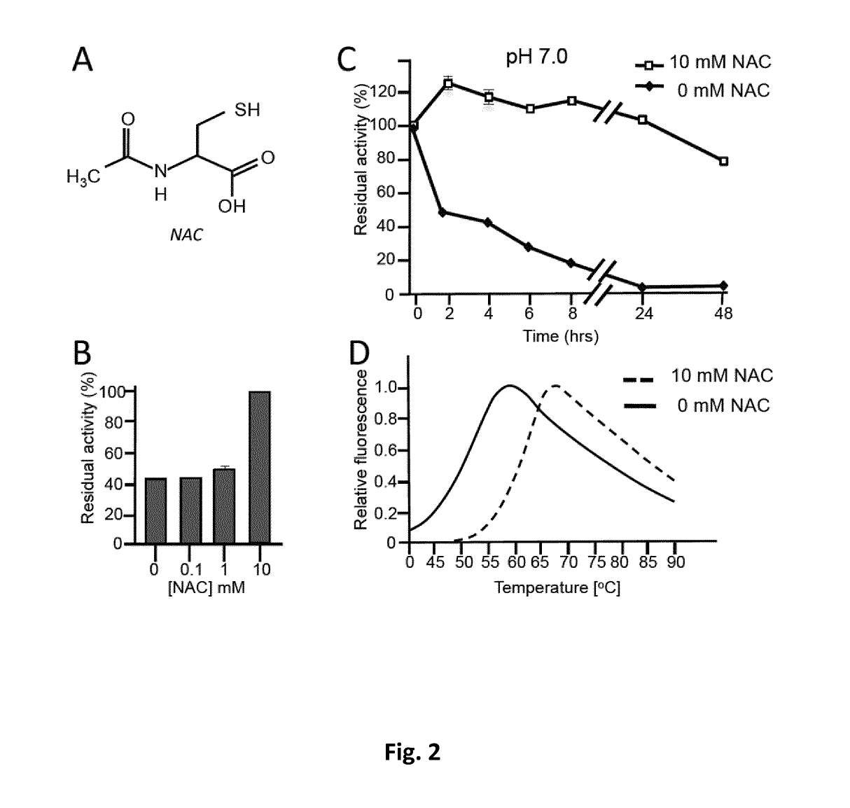

FIG. 2. Effect of NAC on rhGAA stability. (A) NAC structure; (B) NAC was incubated with rhGAA at three concentrations (0, 0.1, 1, 10 mM) with the highest stabilizing effect observed at 10 mM concentration. (C) At the concentration of 10 mM, about 90% of the activity was detectable even after 48 h of incubation. (D) rhGAA thermal stability: changes in the fluorescence of SYPRO Orange was monitored as a function of temperature at pH 7.4.

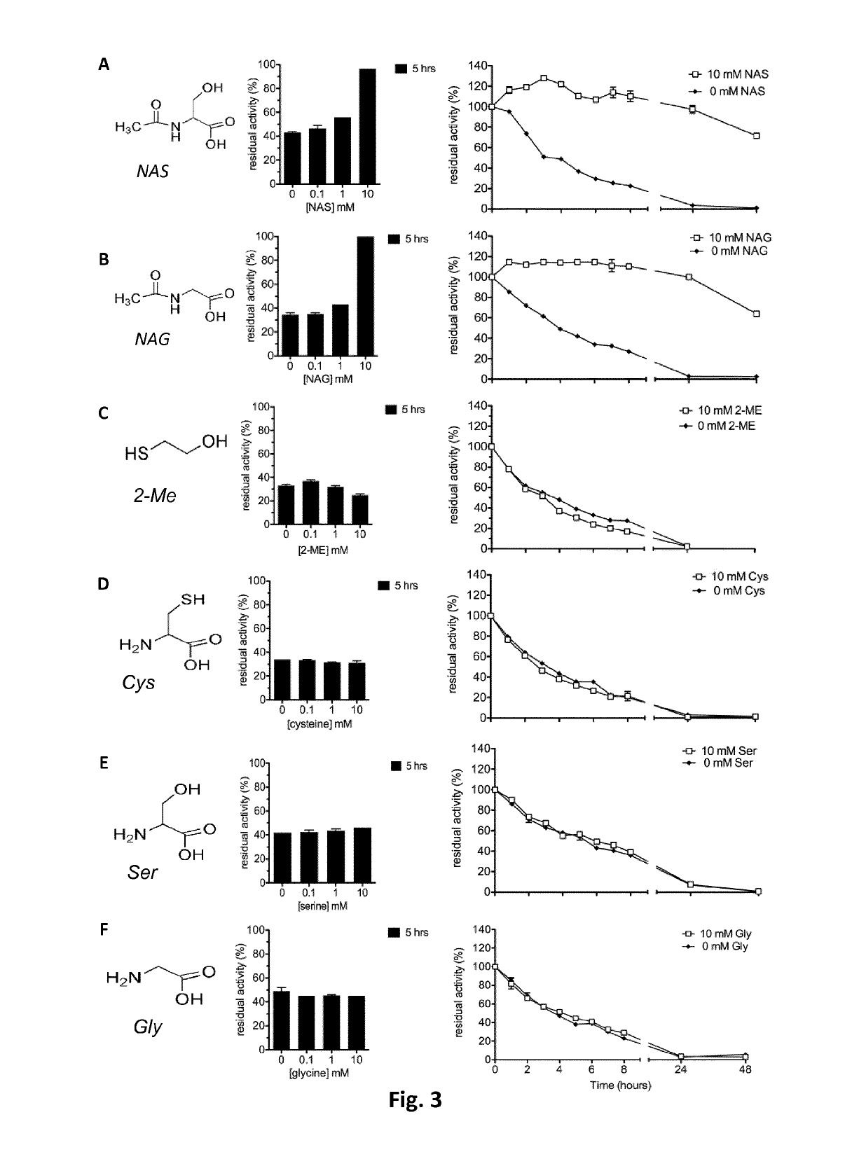

FIG. 3. Effect of NAS, NAG and non-acetylated amino acids on rhGAA. The effect on the rhGAA activity at different concentrations (0, 0.1, 1, 10 mM) and up to 48 hrs of incubation was performed by using (A, B) acetylated amino acids NAS and NAG, (D-F) non-acetylated homologs Cys, Ser, and Gly, and (C) 2-mercapto-ethanol.

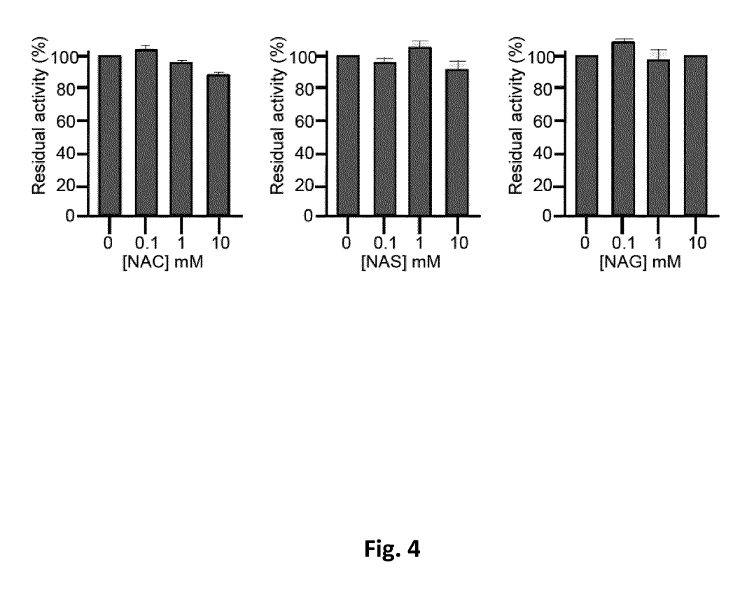

FIG. 4. Comparison of the effect of NAC, NAS, and NAG on GAA activity at three concentrations.

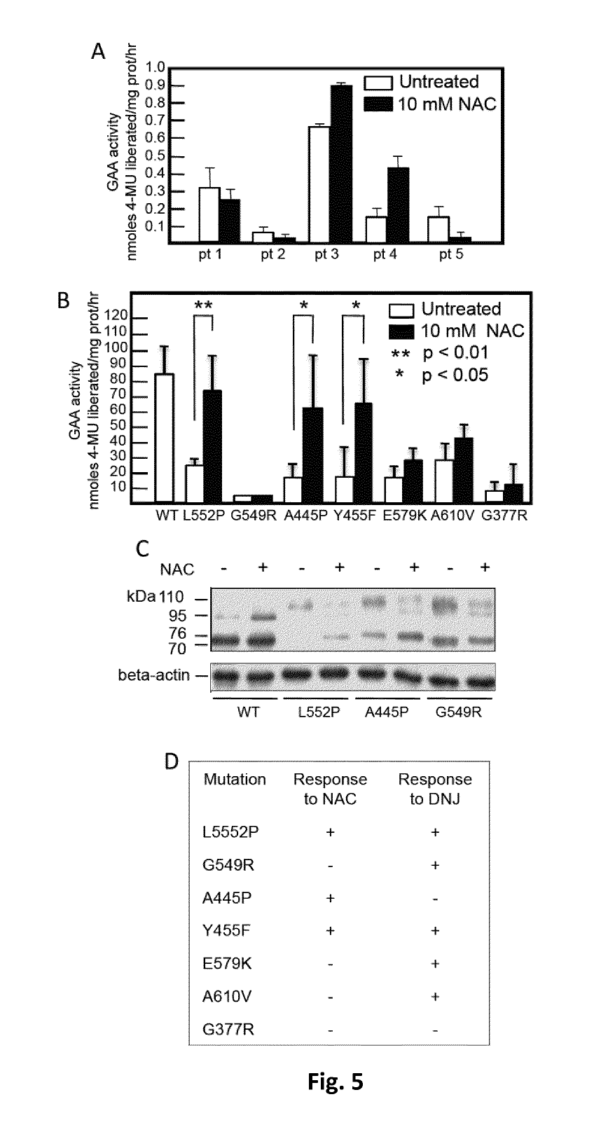

FIG. 5. Effect of NAC on the residual activity of mutated GAA in fibroblasts and COS7 cells. (A) Five fibroblast cell lines from PD patients were incubated with 10 mM NAC for 24 hrs and the activity was assayed in cell homogenates. NAC enhanced the residual GAA activity in 3 out of 5 cell lines studied (derived from patients 1, 3 and 4). (B) The response of mutated GAA to NAC was also evaluated by expressing a panel of mutated GAA gene constructs in COS7 cells. The mutations p.L552P, p.A445P, p.Y455F, p.E579K, showed increases of GAA activity indicating that NAC can rescue in part the residual activity of mutated GAA. (C) In the presence of NAC the amounts of 76 and 70 kDa active GAA isoforms analyzed by western blot increased in cellular extracts from COS7 cells over-expressing responsive (p.L552P and p.A445P) mutated constructs. (D) NAC has a different chaperoning profile compared to the active site-directed chaperones DNJ.

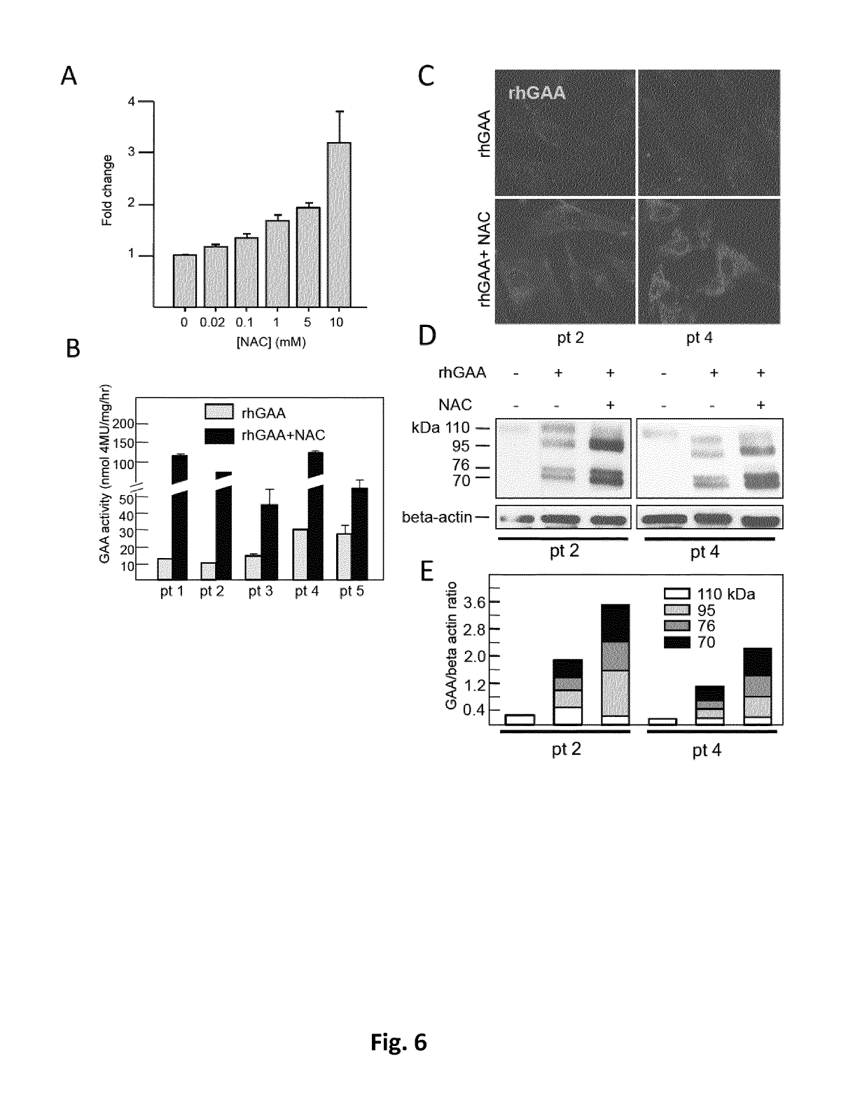

FIG. 6. Synergy between NAC and rhGAA in PD fibroblasts. (A) The efficacy of rhGAA was enhanced by different concentrations (0.02-10 mM) of NAC in patient 3, showing a dose-dependent effect. (B) Five PD fibroblast cell lines were incubated with 50 μM rhGAA in the absence and presence of 10 mM NAC. In all cell lines co-incubation of rhGAA with the chaperone resulted in an improved correction of GAA deficiency, with increases in GAA activity ranging from approximately 3.7 to 13.0-fold the activity of cells treated with rhGAA alone. (C) In the presence of NAC also the amount of fluorochrome-labelled GAA increased (light gray), compared to cells incubated with fluorescent GAA alone. (D) The amounts of GAA polypeptides in the cells treated with NAC and rhGAA were also increased, compared to cells treated with the recombinant enzyme alone. (E) The relative amounts of mature active GAA isoforms of 76 and 70 kDa was increased in the presence of NAC.

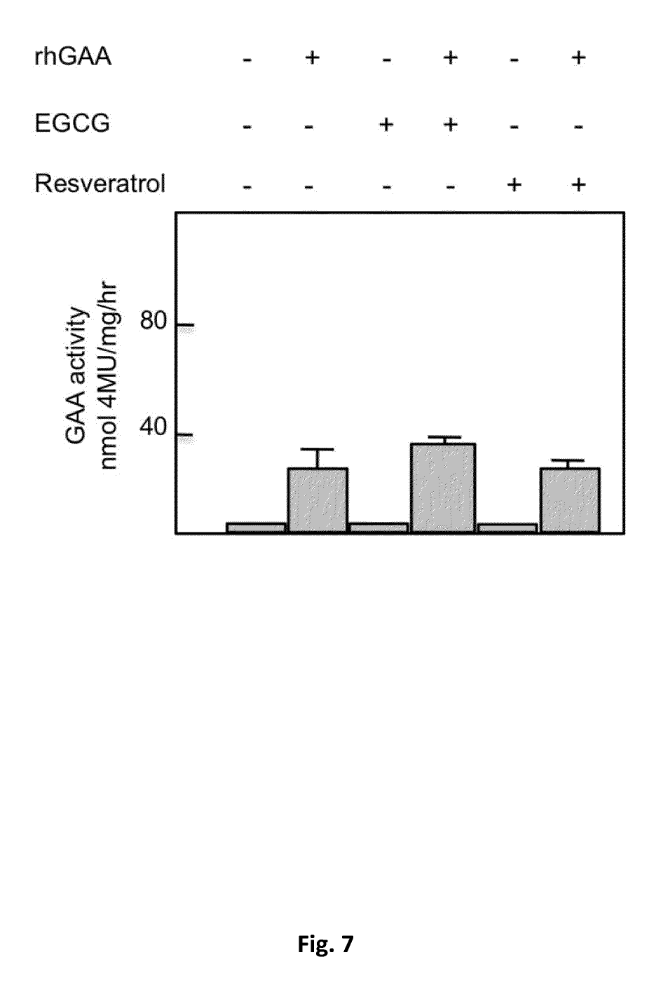

FIG. 7. Effect of the antioxidants epigallo catechingallate (EGCG) and resveratrol on the efficacy of rhGAA in cultured PD fibroblasts (patient 3). Neither of the two drugs showed enhancement of rhGAA.

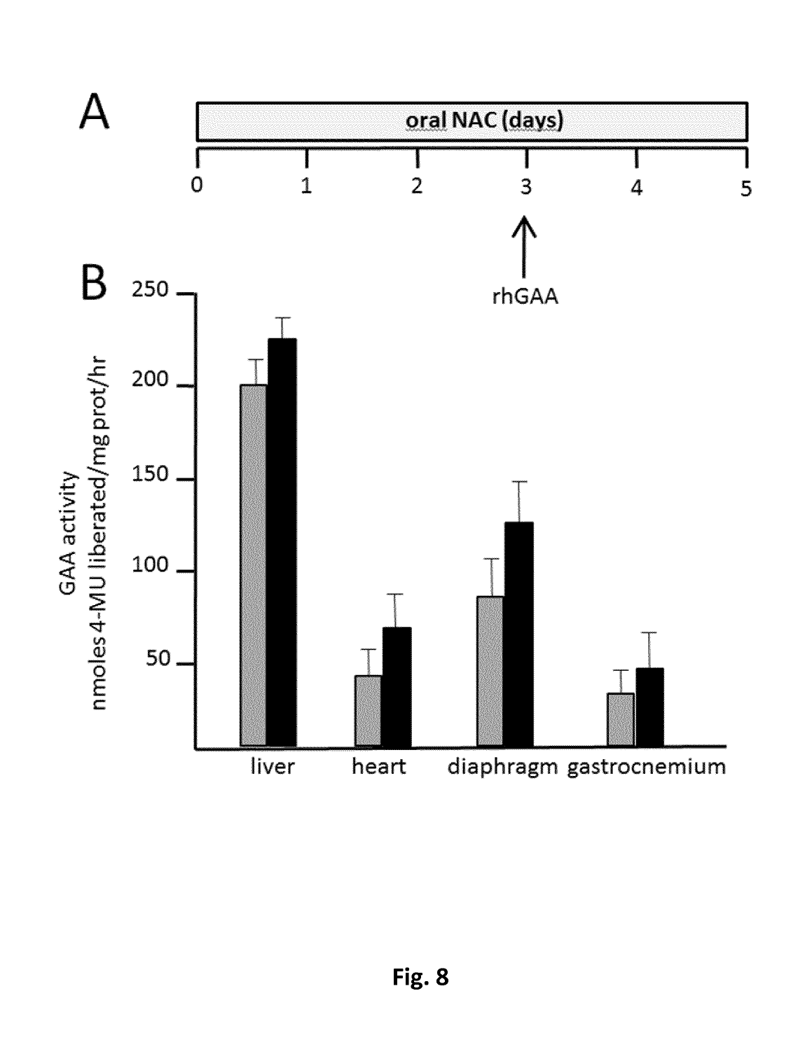

FIG. 8. Synergy between NAC and rhGAA in vivo. (A) Mice were treated with oral NAC for 5 days and received an rhGAA injection on day 3. Animal treated with rhGAA alone were used as controls. (B) In all tissues examined (liver, heart, diaphragm and gastrocnemium) the combination of NAC and rhGAA (black bars) resulted in higher GAA enzyme activity compared to rhGAA alone (grey bars).

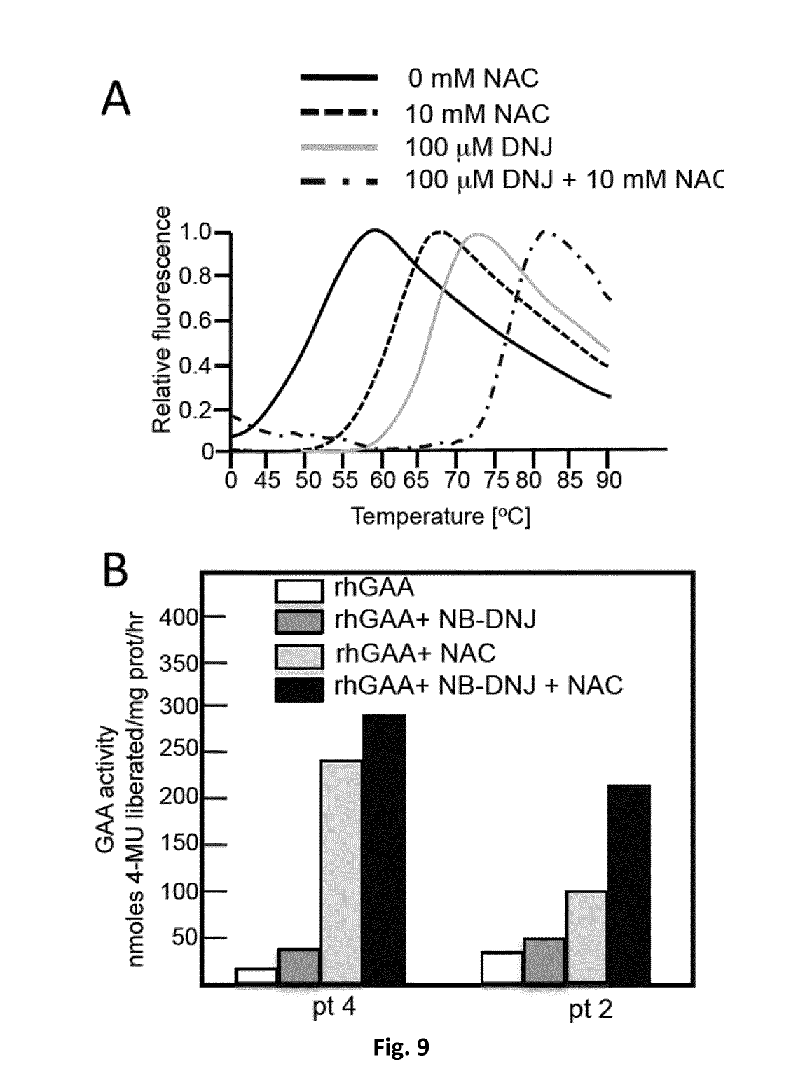

FIG. 9. Comparison of the effect of NAC with NB-DNJ. (A) Thermal stability scans of rhGAA were performed in the absence and in the presence of NAC or NB-DNJ. Both chaperones increased thermal stability of rhGAA, with NB-DNJ resulting in the best shift in Tm (65.9±0.3° C.) of the enzyme. (B) PD fibroblasts from patients 2 and 4 were treated with rhGAA, with rhGAA plus either NAC or NB-DNJ, and with rhGAA plus the combination of the two chaperones. In both cell lines the combination of NAC and NB-DNJ resulted in the highest enhancement of GAA activity by rhGAA.

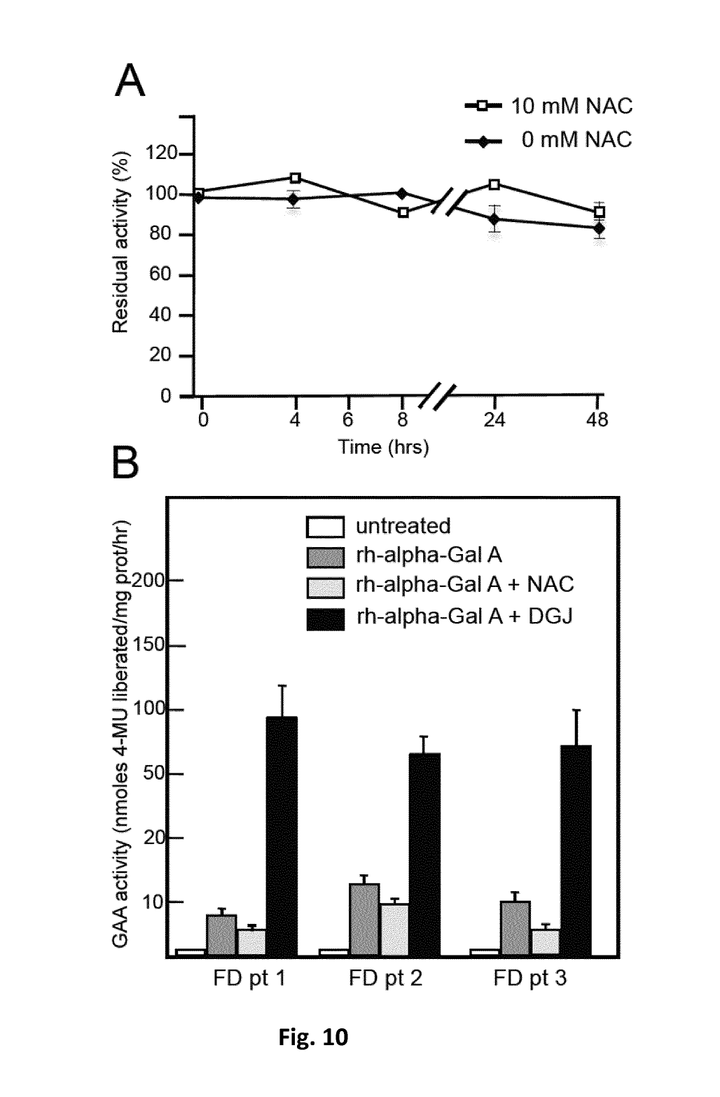

FIG. 10. Effect of NAC on rh-alpha-Gal A. (A) rh-alpha-Gal A was incubated in 50 mM sodium citrate/phosphate buffer at neutral pH 7.0, in the presence or in the absence of 10 mM NAC. The chaperone had no effect on rh-alpha-Gal A after 48 h. (B) Three Fabry disease cell lines were incubated with rh-alpha-Gal A, in the absence and in the presence of NAC, and in the presence of the known chaperone DGJ. NAC had no enhancing effect on the correction of alpha-deficiency by rh-alpha-Gal A in the cells studied. As expected DGJ largely enhanced the effects of rh-alpha-Gal A.

### DETAILED DESCRIPTION OF THE INVENTION

### Materials and Methods

### Fibroblast Cultures

Fibroblasts from PD and Fabry disease patients were derived from skin biopsies after obtaining the informed consent of patients. Normal age-matched control fibroblasts were available in the laboratory of the Department of Pediatrics, Federico II University of Naples. All cell lines were grown at 37° C. with 5% CO2 in Dulbecco's modified Eagle's medium (Invitrogen, Grand Island, N.Y.) and 10% fetal bovine serum (Sigma-Aldrich, St Louis, Mo.), supplemented with 100 U/ml penicillin and 100 mg/ml streptomycin.

### Reagents

rhGAA (alglucosidase, Myozyme) and rh-alpha-Gal A (agalsidase-beta, Fabrazyme) were from Genzyme Co, Cambridge, Mass., USA. Enzymes were prepared and diluted according to manufacturer instructions to NAC, NAS, NAG, Cys, Ser, Gly, 2-mercaptoethanol, 4-nitrophenyl-α-glucopyranoside (4NP-Glc) NB-DNJ and DGJ were from Sigma-Aldrich.

Epigallo catechingallate (Cat. No. 93894) and Resveratrol (Cat. No. 34092) were purchased from Sigma-Aldrich.

The rabbit anti GAA primary antibody used for immunofluorescence and western blot analysis was purchased from Abnova, Heidelberg, Germany; the anti-beta-actin mouse monoclonal antibody was from Sigma-Aldrich. Anti-rabbit and anti-mouse secondary antibodies conjugated to Alexa Fluor 488 or 596 were from Molecular Probes, Eugene, Oreg.; HRP-conjugated anti-rabbit or anti-mouse IgG were from Amersham, Freiburg, Germany. Labeling of rhGAA was performed using the Alexa Fluor 546 labeling kit (Molecular Probes) according to the manufacturer instructions.

Thermal Stability of rhGAA

Thermal stability scans of rhGAA were performed as described in [Flanagan et al. 2009]. Briefly, 2.5 μg of enzyme were incubated in the absence and in the presence of NAC and DNJ, 10 mM and 0.1 mM, respectively, SYPRO Orange dye, and sodium phosphate 25 mM buffer, 150 mM NaCl, pH 7.4 or sodium acetate 25 mM buffer, 150 mM NaCl, pH 5.2. Thermal stability scans were performed at 1° C./min in the range 25-95° C. in a Real Time LightCycler (Bio-Rad). SYPRO Orange fluorescence was normalized to maximum fluorescence value within each scan to obtain relative fluorescence. Melting temperatures were calculated according to (Niesen et al. 2007).

### Enzyme Characterization

The standard activity assay of rhGAA was performed in 200 μl by using 5 μg of enzyme at 37° C. in 100 mM sodium acetate pH 4.0 and 20 mM 4NP-Glc. The reaction was started by adding the enzyme; after suitable incubation time (1-2 min) the reaction was blocked by adding 800 μL of 1 M sodium carbonate pH 10.2. Absorbance was measured at 420 nm at room temperature, the extinction coefficient to calculate enzymatic units was 17.2 mM⁻¹ cm⁻¹. One enzymatic unit is defined as the amount of enzyme catalyzing the conversion of 1 μmol substrate into product in 1 min, under the indicated conditions.

The effect of different pHs on the rhGAA stability was measured by preparing reaction mixtures containing 0.75 mg mL⁻¹ of enzyme in the presence of 50 mM citrate/phosphate (pH 3.0-7.0) at a certain pH. After incubations at 37° C., aliquots were withdrawn at the times indicated and the residual α-glucosidase activity was measured with the standard assay. To test the effect on the pH stability of rhGAA of chemical chaperons and of the other molecules, experiments were performed as described above by adding to the reaction mixtures the amounts of the different compounds indicated in the text.

Incubation of Fibroblasts with rhGAA and GAA Assay

To study the rhGAA uptake and correction of GAA activity in PD fibroblasts, the cells were incubated with 50 micromol/1 rhGAA for 24 hours, in the absence or in the presence of 10 mM NAC. Untreated cells or were used for comparison. After the incubation the cells were harvested by trypsinization and disrupted by 5 cycles of freezing and thawing.

GAA activity was assayed by using the fluorogenic substrate 4-methylumbelliferyl-alpha-D-glucopyranoside (Sigma-Aldrich) according to a published procedure [Porto et al, 2009]. Briefly, 25 micrograms of protein were incubated with the fluorogenic substrate (2 mM) in 0.2 M acetate buffer, pH 4.0, for 60 minutes in incubation mixtures of 100 μl. The reaction was stopped by adding 700 μl of glycine-carbonate buffer, pH 10.7. Fluorescence was read at 365 nm (excitation) and 450 nm (emission) on a Turner Biosystems Modulus fluorometer. Protein concentration in cell homogenates was measured by the Bradford assay (Biorad, Hercules, Calif.).

### Western Blot Analysis

To study GAA immunoreactive material, fibroblast extracts were subjected to western blot analysis. The cells were harvested, washed in phosphate-buffered saline, resuspended in water, and disrupted by five cycles of freeze-thawing. Equal amounts (20 μg protein) of fibroblast extracts were subjected to sodium dodecyl sulfate polyacrylamide gel electrophoresis and proteins were transferred to PVD membrane (Millipore, Billerica, Mass.). An anti-human GAA antiserum was used as primary antibody to detect GAA polypeptides; to detect β-actin, a monoclonal mouse antibody was used Immunoreactive proteins were detected by chemiluminescence (ECL, Amersham, Freiburg, Germany)

### Immunofluorescence Analysis and Confocal Microscopy

To study the distribution of AlexaFluor546 labeleld GAA, PD fibroblasts grown on coverslips were fixed using methanol, permeabilized using 0.1% saponin and locked with 0.01% saponin, 1% fetal bovine serum diluted in phosphate-buffered saline for 1 hour. The cells were incubated with the primary antibodies, with secondary antibodies in blocking solution and then mounted with vectashield mounting medium (Vector Laboratories, Burlingame, Calif.). Samples were examined with a Zeiss LSM 5 10 laser scanning confocal microscope. Authors used Argon/2 (458, 477, 488, and 514 nanometers) and HeNe1 (543 nanometers) excitation lasers, which were switched-on separately to reduce crosstalk of the two fluorochromes. The green and the red emissions were separated by a dichroic splitter (FT 560) and filtered (515-540-nm bandpass filter for green and >610-nm long pass filter for red emission). A threshold was applied to the images to exclude ˜99% of the signal found in control images.

### In Vivo Studies

Animal studies were performed according to the European Union Directive 86/609, regarding the protection of animals used for experimental purposes. The animals, mice model of PD [Raben et al, 1993], were allowed to drink 138 mM NAC in water ad libitum (4.2 g/kg/day) for 5 days. On day 3 they received a single rhGAA injection (100 mg/kg) in the tail vein. On day 5 the animals were sacrificed and tissues were analyzed for GAA activity.

### Results

NAC Improves rhGAA Stability In Vitro

Previous studies have shown that changes of the physical environment, such as modifications of temperature and pH, induce perturbations in the conformation of lysosomal enzymes and affect their stability [Liebermann et al, 2007; Shen et al, 2008]. Resistance of wild-type enzymes to these physical stresses is commonly taken as an indicator to monitor the efficacy of pharmacological chaperones [Valenzano et al, 2011]. The effect of pH on rhGAA stability is shown in FIG. 1.

At pH 5.0 rhGAA was stable for up to 24 hours. At non-lysosomal pH, either acidic (3.0) or neutral (7.0, representative of non-lysosomal cellular compartments), the enzyme was unstable and rapidly lost its activity with approximately 50% residual activity after 4 hours and near complete inactivation (less than 10% residual activity) after 16 hours.

The stability of rhGAA at neutral pH was rescued by co-incubation with NAC (FIG. 2A). The stabilizing effect of NAC on rhGAA was dose dependent (FIG. 2B), and maintained even after 48 h of incubation (FIG. 2C).

NAC increased also the rhGAA thermal stability: at 10 mM concentration the melting temperature (Tm) of rhGAA increased by 10.5±0.5° C. (Tm 60.7±0.5° C. vs 50.2±0.1° C.) (FIG. 2D).

To test whether the stabilizing effect of NAC resulted from the sulfidryl group, the related amino acids N-acetyl serine (NAS) and N-acetyl glycine (NAG) were also tested. Both compounds behaved as NAC by inducing remarkable stabilization of rhGAA at pH 7.0 (FIGS. 3A and 3B) with no effect on the activity of the enzyme (FIG. 4).

Thus, these molecules, binding GAA at an allosteric sites that is different than the protein's active site, belong to a new class of allosteric non-inhibitory chaperones.

The non-acetylated homologs Cys, Ser, and Gly and 2-mercapto-ethanol, a structurally unrelated compound containing an SH group, did not prevent enzyme inactivation (FIG. 3C-F). These data indicate that the stabilizing effect was due to the presence of the acetyl group rather than to the sulfidryl group. Thus, the —SH group may be substituted by different functionalities without abrogating the binding of the small-molecule to the enzyme.

### NAC Rescues Mutated GAA in PD Fibroblasts and Transfected COS Cells

Authors investigated the effect of NAC in cultured fibroblasts from five PD patients (pt) carrying different mutations and with different phenotypes (see Table II). In these studies authors focused on NAC as this molecule is already approved for clinical use and thus has a greater potential for clinical translation compared to NAS and NAG.

TABLE II
PD fibroblast cell lines studied

| | | | Average | |
|---|---|---|---|---|
| | | | GAA | Studies in which |
| | | | residual | the same cell |
| pt | Phenotype | Genotype* | activity** | line was used |
| pt 1 | Severe | p.W367X/ | 0.20 | Parenti et al, 2007; |
| | | p.G643R | | Cardone et al, 2008; |
| | | | | Porto et al, 2009) |
| pt 2 | Severe | p.H612_ | 0.14 | not reported in |
| | | D616del- | | previous studies |
| | | insRGI/ | | |
| | | p.R375L | | |
| pt 3 | Intermediate | p.L552P/ | 0.69 | Rossi et al, 2007; |
| | | aberrant | | Cardone et al, 2008; |
| | | splicing | | Porto et al, 2009) |
| pt 4 | Intermediate | c.-32- | 0.15 | not reported in |
| | | 13T > G - | | previous studies |
| | | p.S619N/ | | |
| | | p.L552P | | |
| pt 5 | Intermediate | G549R/ | 0.16 | Rossi et al, 2007; |
| | | aberrant | | Cardone et al, 2008; |
| | | splicing | | Porto et al, 2009) |

*The genotype of patients was obtained as a routine diagnostic procedure to confirm the diagnosis of Pompe disease. Patients or their legal guardians provided their informed consent for the molecular analysis of the GAA gene. Pt 2 on one allele has a deletion from amino acid residue 612 (histidine) to 616 (aspartate) and insertion of RGI (arginine-glycine-isoleucine); on the 5 second allele mutation Arginine3751eucine.
Pt 4: three mutations splicing c-32-13T > G and p.S619N (in cis); on the second allele p.L5552P
PER FAVORE INSERIRE INFO PER GENOTIPO pt 2 e pt 3
**Activity measured in fibroblasts and expressed as nmoles of 4-methylumbelliferyl-alpha-D-glucopyranoside (4MU) liberated/mg protein/hr (control values 58.5 ± 28.1 nmoles 4MU/mg protein/hr).

NAC enhanced the residual activity of mutated GAA in fibroblasts from patients 3 and 4 (FIG. 5A). Patient 3 had the mutation L552P in association with a mutation causing aberrant splicing. Patient 4 carried three mutations (two, c.-32-13T and p.S619N, on one allele, and the p.L552P mutation on the other allele). Of these mutations the p.L552P, has been previously reported to be responsive to DNJ [Parenti et al, 2007; Flanagan et al, 2009].

The response of individual mutations to NAC was further evaluated by expressing a panel of mutated GAA gene constructs in COST cells, according to the methods reported in previous studies [Parenti et al, 2007; Flanagan et a, 2009] (FIG. 5B). The mutated constructs were chosen to be representative of both imino sugar-responsive and non-responsive mutations, in order to compare the chaperoning profile of NAC with that of imino sugars. The cells were transfected with the mutated constructs, incubated either in the presence or in the absence of 10 mM NAC and harvested 72 hours after transfection. The mutations p.L552P, p.A445P, and p.Y455F showed significant (p<0.01 and p<0.05 for L552P and for A445P and Y455F, respectively), enhancement of GAA activity in the presence of NAC. The increase in activity of the mutation pG377R was not statistically significant.

The enhancement of enzyme activity in responsive mutations paralleled the increase in either 76 KDa or in 70 kDa active isoforms of GAA on western blot analysis. FIG. 5C shows western blots of COST cells over-expressing two of the responsive (p.L552P, p.A445P). The result of western blot analysis of a non-responsive (p.G549R) mutation is shown for comparison. For this latter mutation no change was seen in the amounts of the GAA active isoforms, already detectable in the absence of NAC (as previously reported in Flanagan et al, 2009). These results suggest that NAC has a different chaperoning profile compared to the active site-directed chaperones DGJ and NB-DNJ (FIG. 5D).

NAC Enhances rhGAA Efficacy in PD Fibroblasts

Authors have previously shown that chaperones enhance the efficacy of wild-type recombinant enzymes in PD and Fabry disease [Porto et al, 2009; Porto et al, 2011]. In PD fibroblasts the imino sugar NB-DNJ enhanced rhGAA efficacy by approximately 1.3 to 2-fold. This effect is of great interest as a possible strategy to improve ERT efficacy in PD, and possibly in other LSDs.

Authors tested whether the allosteric chaperone NAC also show the same synergy. In fibroblasts from patient 3, co-administration of rhGAA and NAC (0.02-10 mM) resulted in improved GAA activity with a dose-dependent effect (FIG. 6A). Authors incubated five PD fibroblast cell lines with 50 microM rhGAA in the absence and presence of NAC. The efficacy of rhGAA in correcting the enzymatic deficiency varied among the different cell lines, as already reported in previous papers [Cardone et al, 2008; Porto et al, 2009], possibly due to individual factors implicated in the uptake and intracellular trafficking of the recombinant enzyme in each cell line. However, in all cell lines tested, co-incubation of rhGAA with NAC resulted in an improved correction of GAA deficiency, with increases in GAA activity ranging from approximately 3.7 to 13.0-fold with respect to the activity of cells treated with rhGAA alone (FIG. 6B).

The enhancing effect largely exceeded that due to the rescue of the native mutated enzyme (patients 3 and 4) and was observed also in non-responsive cell lines (patients 2 and 5). Authors also observed an increase in the amount of fluorochrome-labelled GAA in the presence of the chaperone NAC, compared to cells incubated with fluorescent GAA alone (FIG. 6C). By this approach only the fluorescent exogenous enzyme is detectable and variations in the intensity of fluorescence reflect only the effects on the recombinant enzyme. The combination of these results supports the concept that the enhancing effect of chaperones is directed towards the wild-type recombinant enzyme and is consistent with the data previously reported with the chaperone NB-DNJ [Porto et al, 2009].

A western blot analysis (FIG. 6D) and the quantitative analysis of each band (FIG. 6E) showed increased amounts of GAA-related polypeptides in the cells treated with NAC and rhGAA, compared to cells treated with the recombinant enzyme alone. The processing of rhGAA (that is provided in commercial preparations for clinical use as 110 kDa precursor) into the mature active isoforms was also improved in the presence of NAC. The analysis of the GAA band density showed a relative increase of the intermediate (95 kDa) and mature (76-70 kDa) GAA molecular forms in the presence of NAC, compared to the 110 kDa precursor. Since the GAA precursor is converted into the active forms in the late-endosomal/lysosomal compartment [Wisselaar et al, 1993], this indicates improved lysosomal trafficking of the enzyme.

Other anti-oxidant drugs (resveratrol, epigallo chatechingallate) did not enhance rhGAA in PD cultured fibroblasts (FIG. 7). These results, together with the analysis of NAC-GAA interaction and with the data in cell-free systems, exclude that the effect of NAC is due to its anti-oxidant properties. Authors also tested the combination of NAC and rhGAA in a mouse model of PD [Raben et al, 1993]. Mice were treated with a single injection of rhGAA at high doses (100 mg/kg) in combination with oral NAC for 5 days (FIG. 8A). Mice treated with the recombinant enzyme alone were used as controls. Forty-eight hours after rhGAA injection the animals were euthanized and GAA activity was assayed in different tissues. Albeit not statistically significant, in all tissues examined (liver, heart, diaphragm and gastrocnemium) the combination of NAC and rhGAA was superior to rhGAA alone in correcting enzyme activity (FIG. 8B).

Comparison of NAC with the Imino Sugar Chaperone NB-DNJ, and Specificity of NAC's Effect

To compare the effect of NAC on thermal denaturation of rhGAA to that of the imino sugar DNJ, authors performed thermal stability scans of rhGAA in the absence and in the presence of the two chaperones. Both chaperones increased rhGAA thermal stability, with DNJ causing the best shift in Tm (65.9±0.3° C.). This result is apparently in contrast with the data shown in FIG. 7, indicating that NAC is superior to imino sugars in enhancing the efficacy of rhGAA in PD fibroblasts. The discrepancy of these results, however, may be explained by the lack of inhibitory effect of NAC on the recombinant enzyme in cells.

A corollary of the fact that NAC and imino sugar chaperones interact with different protein domains, is that their effect may be cumulated. This might represent an additional advantage for the treatment of patients, in order to obtain the best stabilization of rhGAA and the highest synergy with ERT. This hypothesis was supported by the results of thermal denaturation of rhGAA, showing the highest stability of the enzyme with the combination of NAC and DNJ (Tm=75.9±0.3) (FIG. 9A), and by studies in two PD cell lines (from patients 2 and 4). These cells were incubated with rhGAA, with rhGAA plus either NAC or NB-DNJ, and with rhGAA plus the combination of the two chaperones. In both cell lines the combination of NAC and NB-DNJ resulted in the highest enhancement of GAA activity by rhGAA (FIG. 9B).

An important concern on the use of pharmacological chaperones is the specificity of their effects and the possibility of interactions with other enzymes. GAA belongs to family GH31 of glycoside hydrolases, interestingly, this family was included in the GH-D superfamily of glycoside hydrolases together with families GH36 and GH27 [Ernst et al. 2006]. The latter family includes lysosomal alpha-galactosidase A (alpha-Gal A), that is defective in another LSD, Fabry disease [Germain, 2010]. Two preparations of recombinant human alpha-Gal A (rh-alpha-Gal A) have been approved for ERT in Fabry disease patients. To test if NAC is active on this enzyme, authors incubated rh-alpha-Gal A and 10 mM NAC in 50 mM sodium citrate/phosphate buffer, pH 7.0. NAC had no effect on rh-alpha-Gal A after 48 h (FIG. 10A).

In addition, when authors incubated three Fabry disease cell lines with rh-alpha-Gal A, in the absence and in the presence of NAC, and in the presence of the known chaperone DGJ (FIG. 10B), NAC had no enhancing effect on the correction of alpha-deficiency by rh-alpha-Gal A. As expected and as shown in a previous study where the same cell lines were used [Porto et al, 2011] DGJ largely enhanced the effects of rh-alpha-Gal A.

### DISCUSSION

Therapeutic strategies directed towards the rescue of defective mutant enzymes are an attractive alternative to therapies based on the supply of wild-type enzyme, such as ERT, gene therapy and transplantation of hematopoietic stem cells. The rescue of the mutant enzyme may be obtained by various approaches. One is aimed at adjusting with small-molecule drugs the capacity of the cellular networks controlling protein synthesis, folding, trafficking, aggregation, and degradation, thus facilitating the exit of mutated proteins from the endoplasmic reticulum [Mu et al, 2008; Powers et al, 2009; Ong and Kelly, 2011; Wang et al, 2011].

Alternatively, small-molecule drugs, so called pharmacological chaperones, may act directly on the defective enzymes, favoring the most stable conformation(s) of these proteins, and preventing their recognition and disposal by the endoplasmic reticulum associated quality control and degradation systems.

Albeit attractive and rapidly evolving towards clinical translation, some aspects of the biochemistry of PCT are incompletely understood or require optimization. An important issue in this respect is the potential inhibition of target enzymes. According to a recent review all chaperones proposed or used for the treatment of LSDs are reversible competitive inhibitors of the target enzymes, and may in principle interfere with the activity of these enzymes [Valenzano et al, 2011]. Thus, treatment protocols based on the pulsed administration of chaperones (that have a short plasma half-life) to rescue mutant enzymes (that in general have a longer half-life) have been so far developed and tested in LSDs mouse models [Khanna et al, 2010; Benjamin et al, 2012].

Another limitation of chaperones is that they are effective in rescuing only some disease-causing missense mutations, mainly located in specific enzyme domains, and are thus potentially effective only in a limited number of patients. For PD, it is possible to speculate that about 10-15% patients may be amenable to PCT with the imino sugar DNJ [Flanagan et al, 2009].

These problems can be addressed by the identification of novel and allosteric non-inhibitory chaperones. In this study, authors have shown that NAC and the related compounds NAS and NAG, have these features, being able to stabilize GAA without interfering with its activity and having a different chaperoning profile, compared to known chaperones. NAC is a known anti-oxidant that was evaluated in the authors' laboratory, together with other related drugs (resveratrol, epigallo catechingallate) in PD fibroblasts for possible effects on rhGAA intracellular trafficking. The characterization of NAC's mechanism of action on rhGAA, however, indicated that molecular interactions with the enzyme, rather than the anti-oxidant effect, were responsible for rhGAA stabilization and that the other anti-oxidants studied did not stabilize the enzyme. This was somewhat surprising because NAC is structurally very different from the imino sugars, the only known pharmacological chaperones of GAA so far, that resemble the natural substrates/products of the enzyme.

Authors showed that NAC improved stability of GAA in response to physical stresses. For instance, increased resistance to pH variations is particularly interesting. Compared to methods based on temperature denaturation, which are often used as a measure of the effects of chaperones, neutral pH may be more representative of some of the environmental conditions encountered by recombinant enzymes in plasma and in certain cellular compartments. It has been shown that pH induces conformational changes in lysosomal enzymes. This has been studied in detail for GBA [Lieberman et al, 2007; Lieberman et al, 2009]. GBA stability and conformation were analyzed in neutral and in acidic pH environments, and in complex with the pharmacological chaperone IFG. Changes in pH resulted in different conformations of the enzyme, with small but critical differences in two loops localized at the mouth of active site. IFG binding favored the most stable conformations of the enzyme [Lieberman et al, 2007].

In cell-free assays NAC prevented the loss of GAA activity as a function of pH and increased the enzyme thermal stability. In COST cells overexpressing mutated GAA incubation with NAC resulted in increased residual GAA activity for four of the seven mutations studied. Remarkably, the chaperoning profile of NAC showed differences compared to that of NB-DNJ and DGJ. The mutation p.A445P, previously reported as non-responsive to imino sugar chaperones, appeared to be responsive to NAC. This may translate into an expansion of the number of chaperone-responsive mutations, and should be further investigated in large-scale studies, like that performed in 76 different variants of the GAA gene [Flanagan et al, 2009]. It may be envisaged that preliminary screenings in vitro on a number of chaperones would allow personalization of treatment protocols aimed at obtaining the greatest beneficial effect in different PD patients. In these regards, the identification of NAC and derivatives, which are structurally very different from the other known pharmacological chaperones identified in PD is quite promising. In fact, other molecules, whose chaperoning activity cannot be simply inferred from their structure, may be effective in several LSD, thereby opening new and wider opportunities for the identification of novel therapeutic drugs.

NAC also increased the efficacy of recombinant GAA, in particular rhGAA, in correcting the enzyme defect in PD fibroblasts. Compared to the effect of NAC, and of chaperones in general, on the mutated enzymes, this effect holds greater promise for the cure of patients affected by PD, and possibly of other LSDs. It should be considered that, while the enhancement of endogenous defective enzymes by chaperones in most cases resulted in minor changes in terms of residual activity (likely with a modest impact on patients' outcome), the synergy of these drugs with ERT caused (at least in cellular systems) remarkable increases of specific activity. In this study co-administration of NAC and recombinant GAA, in particular rhGAA, resulted in complete correction of the enzymatic defect.

A synergy between chaperones and ERT has already been described using known chaperones in PD and Fabry disease [Shen et al, 2008; Porto et al, 2009; Porto et al, 2011; Benjamin et al, 2012]. The molecular bases of this synergy, however, are still incompletely understood. The enhancing effect of chaperones on ERT may imply that a substantial fraction of the recombinant enzymes, during their traffic to lysosomes, is prone to degradation and is not able to reach its final destination. For the recombinant human GBA, used for ERT in Gaucher disease, it was suggested that inability to recover most of the infused recombinant enzyme in the target tissues was due to losses occurring during transit to the lysosome [Xu et al, 1996; Shen et al, 2008]. It has also been speculated that factors related to purification steps, body temperature and the neutral pH of blood, may result in stress for the enzyme during its transit through the circulation and tissue fluids, and lead to greater susceptibility to the action of proteases or denaturation. In a recent study on the co-administration of rh-alpha-Gal A in the murine model of Fabry diseases it has been shown that incubation of the recombinant enzyme in blood results in decreased stability [Benjamin et al, 2012]. In principle, the enhancing effect of chaperones on recombinant enzymes may be due to stabilization of the enzyme in the cell medium, to improved uptake by the cells, or to stabilization of the enzyme intracellularly, either through the endocytic pathway or within the lysosomal compartment. The present results showing an enhancing effect of NAC on the mutant enzyme in cultured fibroblasts and in COST cells over-expressing mutated enzymes would favor the hypothesis that, at least in part, the stabilization occurs intracellularly.

The present results support a synergy between chaperones and recombinant enzymes and have important clinical implications and may translate into improved clinical efficacy of ERT, as shown in in vivo experiments in PD mice.

### REFERENCES

Benjamin E R, et al., Mol Ther. 2012 Jan. 3
Beutler E. Mol Genet Metab. 2006 July; 88(3):208-15
Cardone M, et al., Pathogenetics. 2008 Dec. 1; 1(1):6.
Chien Y H, et al., Pediatrics. 2009 December; 124(6):e1116-25
Ernst H A, et al. J Mol Biol 2006 May 12; 358(4) 1106-24.
Fan J Q. Biol Chem. 2008 January; 389(1):1-11
Flanagan J J, et al., Hum Mutat. 2009 December; 30(12):1683-92.
Fukuda T, et al., Mol Ther. 2006 December; 14(6):831-9
Fukuda T, et al., Ann Neurol. 2006 April; 59(4):700-8
Germain D P. Orphanet J Rare Dis. 2010 Nov. 22; 5:30
Khanna R, et al., Mol Ther. 2010 January; 18(1):23-33
Kishnani P S, et al., Neurology. 2007 Jan. 9; 68(2):99-109.
Kishnani P S, et al., Pediatr Res. 2009 September; 66(3):329-35.
Kishnani P S, et al., Mol Genet Metab. 2010 January; 99(1):26-33
Koeberl D D, et al., Mol Genet Metab. 2011 June; 103(2):107-12
Lieberman R L, et al., Biochemistry. 2009 Jun. 9; 48(22):4816-27.
Lieberman R L et al., Nat Chem Biol. 2007 February; 3(2):101-7
Mu T W, et al., Cell. 2008 Sep. 5; 134(5):769-81
Niesen F H, et al., Nat Protoc. 2007; 2(9):2212-21.
Ong D S, Kelly J W. Curr Opin Cell Biol. 2011 April; 23(2):231-8
Powers E T, et al., Annu Rev Biochem. 2009; 78:959-91.
Parenti G, et al., Mol Ther. 2007 March; 15(3):508-14
Parenti G. EMBO Mol Med. 2009 August; 1(5):268-79
Parenti G, et al., Curr Pharm Biotechnol. 2011 June; 12(6):902-15.
Pollegioni, Loredano; Servi, Stefano (Unnatural Amino Acids, Methods and Protocols Series: Methods in Molecular Biology, Vol. 794; Eds. 2012, XIV, 409p. 123 illus., Humana Press)
Porto C, et al., Mol Ther. 2009 June; 17(6):964-71.
Porto C, et al., J Inherit Metab Dis. 2011 Dec. 21.
Raben N, et al. J Biol Chem. 1993, 273:19086-92.
Raben N, et al., Mol Genet Metab. 2003 September-October; 80(1-2):159-69
Raben N, et al., Autophagy. 2009 January; 5(1):111-3
Schoser B, Hill V, Raben Neurotherapeutics. 2008 October; 5(4):569-78
Shea L, Raben N. Int J Clin Pharmacol Ther. 2009; 47 Suppl 1:S42-7
Shen J S, et al., Biochem Biophys Res Commun. 2008 May 16; 369(4):1071-5
Strothotte S, et al., J Neurol. 2010 January; 257(1):91-7.
Tropak M B, et al., Chem Biol. 2007 February; 14(2):153-64
Urban D J, et al., Comb Chem High Throughput Screen. 2008 December; 11(10):817-24
Valenzano K J, et al., Assay Drug Dev Technol. 2011 June; 9(3):213-35
Van den Hout J M, et al., Pediatrics. 2004 May; 113(5):e448-57
Van den Hout H, et al., Lancet. 2000 Jul. 29; 356(9227):397-8
Van der Ploeg A T et al., Lancet. 2008 Oct. 11; 372(9646):1342-53
Van der Ploeg A T, et al., N Engl J Med. 2010 Apr. 15; 362(15):1396-406.
Wang F, et al., J Biol Chem. 2011 Dec. 16; 286(50):43454-64
Wenk, J, et al., Biochem Int, 1991, 23: 723-731
Wisselaar H A, et al., J Biol Chem. 1993 Jan. 25; 268(3):2223-31
Xu Y H, et al., Pediatr Res. 1996 February; 39(2):313-22
Zheng W, et al., Proc Natl Acad Sci USA. 2007 Aug. 7; 104(32):13192-7

1-13. (canceled)

14. A method of treatment of a pathological condition characterized by a deficiency of the lysosomal acid alpha-glucosidase (GAA) comprising the administration of an effective dose of an allosteric non-inhibitory chaperone of the lysosomal acid alpha-glucosidase (GAA) to a patient in need thereof.

15. The method according to claim 14 wherein the pathological condition characterized by a deficiency of the lysosomal acid alpha-glucosidase (GAA) is a lysosomal storage disease.

16. The method according to claim 15 wherein the lysosomal storage disease is Pompe disease (PD).

17. The method according to claim 14 wherein the allosteric non-inhibitory chaperone is a N-acetylated amino acid.

18. The method according to claim 14 wherein the allosteric non-inhibitory chaperone is selected from the group consisting of: N-acetyl cysteine (NAC), N-acetyl serine (NAS) or N-acetyl glycine (NAG).

19. The method according to claim 14 further comprising the administration of an effective amount of exogenous GAA and/or the administration of an effective amount of an “active site-directed” chaperone.

20. The method according to claim 19 wherein the “active site-directed” chaperone is selected from the group consisting of: N-butyl-deoxynojirimycin (NB-DNJ) or 1-deoxy-nojiirimycin (DNJ).

21. A method for increasing the activity of an endogenous and/or exogenous GAA in an individual suspected of suffering or suffering from a pathological condition characterized by a deficiency of the lysosomal acid alpha-glucosidase (GAA), which comprises administering to the individual an allosteric non-inhibitory chaperone of the lysosomal acid alpha-glucosidase (GAA) in an amount effective to increase activity of the endogenous and/or exogenous GAA in the individual.

22. The method according to claim 21 wherein the endogenous GAA is in a wild type or mutant form and the exogenous GAA is a recombinant GAA.

23. The method according to claim 21 wherein the pathological condition characterized by a deficiency of the lysosomal acid alpha-glucosidase (GAA) is a lysosomal storage disease, preferably Pompe disease.

24. The method according to claim 21 wherein the allosteric non-inhibitory chaperone is a N-acetylated amino acid.

25. The method according to claim 21 wherein the allosteric non-inhibitory chaperone is selected from the group consisting of: N-acetyl cysteine (NAC), N-acetyl serine (NAS) or N-acetyl glycine (NAG).

26-27. (canceled)

28. A method for identifying an allosteric non-inhibitory chaperone for GAA comprising the steps of:
a) labelling NAC and/or NAS and/or NAG chaperone with a fluorophore;
b) adding to said labeled NAC and/or NAS and/or NAG an amount of rhGAA to obtain a basal rhGAA fluorescence;
c) measuring the basal rhGAA fluorescence;
d) adding a test agent;
e) measuring the fluorescence of rhGAA;
f) comparing the fluorescence of rhGAA measured in c) and e);
wherein if a variation of intensity of fluorescence or if a variation of wavelength of fluorescence is observed then the test agent is an allosteric non-inhibitory chaperone for GAA.
